(19) United States
(12) Patent Application Publication
(10) Pub. No.: US 2026/0274806 A1
(43) Pub. Date: Sep. 17, 2026

(54) NOVEL TAG FOR LIQUID-PHASE PEPTIDE SYNTHESIS

(71) Applicant: CORDENPHARMA INTERNATIONAL GMBH, Plankstadt (DE)

(72) Inventors: Matthieu GIRAUD, Plankstadt (DE); Fernando ALBERICIO, Plankstadt (DE); Oleg MARDER, Plankstadt (DE); Laura HELLRIEGEL, Plankstadt (DE); Mareike HELMDACH, Plankstadt (DE); Fabian HOGENKAMP, Plankstadt (DE); Tim KRAMER, Plankstadt (DE)

(73) Assignee: CORDENPHARMA INTERNATIONAL GMBH, Plankstadt (DE)

(21) Appl. No.: 19/166,582

(22) PCT Filed: Mar. 20, 2024
(86) PCT No.: PCT/EP2024/057509

§ 371 Date: Sep. 18, 2025

(30) Foreign Application Priority Data
Mar. 20, 2023 (EP) 23162993.2

(51) Int. Cl. C07D 251/46; C07F 7/18

(52) U.S. Cl. CPC C07D 251/46; C07F 7/1804

(57) ABSTRACT

The present invention relates compound according to formula (I), wherein, X is selected from the group consisting of —O—(CH₂)ₙ—, wherein n is 0 to 4, R¹ and R² are independently selected from the group consisting of C₁₁-C₂₅ alkyl, SP is a spacer, particularly selected from the group consisting of saturated C₄-C₈ alkyl, unsaturated C₄-C₈ alkyl and substituted or unsubstituted C₆-aromatic compound, in particular the substituted C₆-aromatic compound is an alkyl substituted C₆-aromatic compound, Y is a heteroatom, particularly selected from the group consisting of —O— and —NH—, L is a linker for peptide synthesis and m is 0 or 1, particularly m is 1.

### FIELD

The present invention relates to a novel tag for liquid-phase peptide synthesis.

### BACKGROUND

The invention of Solid Phase Peptide Synthesis (SPPS) by Merrifield in 1963 revolutionized peptide chemistry by implementation of a resin, which allows the coupling and deprotection reactions to be carried out in a heterogenous system and therefore removal of the excess reagents and side products by simple washing steps (Merrifield, R. B., J. Am. Chem. Soc. 1963, 85 (14), 2149-2154). Although efficient SPPS demands excess of reagents and large amounts of solvent, the commonly used Fmoc-tBu strategy can be easily scaled-up and automated for large-scale production of peptides. However, in the context of green chemistry the large amount of waste and the use of hazardous solvents, such as DMF, NMP and DCM, in SPPS should be addressed. In contrast, Liquid-Phase Peptide Synthesis (LPPS) represents an approach which is compatible with these demands, since the reactions are carried out in solution using soluble tags attached to the growing peptide chain (for review see: Albericio, F. et. al. Chem. Rev. 2022, 16, 13516-13546). LPPS can be carried out using less solvent and excess of reagents, while also providing the possibility of implementing green solvents, such as tetrahydrofuran (Chiba, K. Tamaki, H. et. Al. Org. Process Res. Dev. 2019, 23, 11, 2576-2581) and cyclopentyl methyl ether (Kubota, H. et. al. Molecules 2021, 26 (12), 3497). Starting from early PEG-based approaches by Mutter and Bayer (Bayer, E. and Mutter, M., Nature 1972, 237, 512-513; Mutter, M. and Bayer, E., Angew. Chem., Int. Ed. Engl. 1974, 13, 88-89), Tamaki et. al. (Tamaki, H. et. al. Bull. Chem. Soc. Jpn. 2001, 74, 733-738) invented substituted hydrophobic benzyl alcohols (HABs) for tag-assisted LPPS, which have been improved by different groups (e.g.: Sunazuka, T. et. al. Tetrahedron 2011, 67, 6633-6643 and Takahashi, D. et. al. Angew. Chem., Int. Ed. 2017, 56, 7803-7807). These works successfully used soluble HAB-tags for the synthesis of small to medium sized peptides using the Fmoc approach, while usually achieving purification of the desired peptide by precipitation or membrane-assisted filtration and in some cases aqueous work-up procedures.

Based on the above-mentioned state of the art, the objective of the present invention is to provide means and methods for a novel tag for peptide synthesis in solution. This objective is attained by the subject-matter of the independent claims of the present specification, with further advantageous embodiments described in the dependent claims, examples, figures and general description of this specification.

### SUMMARY OF THE INVENTION

A first aspect of the invention relates to a compound according to formula (I)

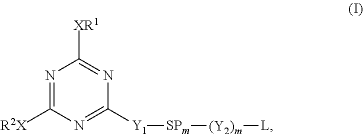

wherein,
X is selected from the group consisting of —O—(CH₂)ₙ—, wherein n is 0 to 4,
R¹ and R² are independently selected from the group consisting of C₁₁-C₂₅ alkyl,
SP is a spacer, particularly selected from the group consisting of saturated C₄-C₈ alkyl, unsaturated C₄-C₈ alkyl and substituted or unsubstituted C₆-aromatic compound, in particular the substituted C₆-aromatic compound is an alkyl substituted C₆-aromatic compound,
Y is a heteroatom, particularly selected from the group consisting of —O— and —NH—,
L is a linker for peptide synthesis, and
m is 0 or 1, particularly m is 1.

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising”, “having”, “containing”, and “including”, and other similar forms, and grammatical equivalents thereof, as used herein, are intended to be equivalent in meaning and to be open-ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a”, “or” and “the” include plural referents unless the context clearly dictates otherwise.

“And/or” where used herein is to be taken as specific recitation of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art (e.g., in cell culture, molecular genetics, nucleic acid chemistry, hybridization techniques and biochemistry, organic synthesis). Standard techniques are used for molecular, genetic, and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The term linker in the context of the present specification relates to a molecule used in Fmoc-Solid Phase Peptide Synthesis (SPPS), which connects the resin on which the SPPS is performed and the forming peptide. The linker may be separated from the peptide through a spacer. The linker is cleaved from the peptide once the desired peptide length is reached.

The term spacer in the context of the present specification relates to a flexible hydrophobic compound consisting of hydrocarbon atoms connecting a linker molecule and the tag core.

The term tag in the context of the present specification relates to a molecule on which a peptide sequence is formed. A tag is usually attached at the N- or C-terminus of the forming peptide.

The formulae of the present specification follow the convention of organic chemistry to not show hydrogen atoms on carbon scaffolds. Carbon is tetravalent and bonds not shown are assumed to be hydrogen unless shown otherwise. Hydrogen can be exchanged for deuterium without changing the bulk chemical properties of the molecule; however, in the case of dye or drug molecules, the exchange of hydrogen for deuterium may lead to changes in the spectral properties or receptor interactions of the molecule. Unless explicitly stated otherwise herein, the disclosure of a formula showing, explicitly or implicitly by the convention restated in the first sentence of this paragraph, encompasses molecules in which one or several of the hydrogen atoms are exchanged for deuterium.

The term saturated alkyl in the context of the present specification relates to a saturated linear or branched hydrocarbon.

The term unsaturated alkyl in the context of the present specification relates to a linear or branched hydrocarbon, wherein one or more carbon-carbon bonds may be unsaturated.

The term alkyl in the context of the present specification relates to a saturated linear, branched or cyclic hydrocarbon consisting of a designated number of carbon atoms. Examples of alkyl residues include but are not limited to n-butyl, iso-butyl, n-pentyl, 2-methylbutyl, 3-methylbutyl, 1,1-dimethylpropyl, 1,2-dimethylpropyl, 1,2-dimethylpropyl, cyclo-pentyl, cyclo-hexyl, methyl-cyclo-pentyl, methyl cyclohexanyl, heptyl, decanyl, dodecyl. In certain embodiments, a C₅ alkyl is a pentyl or cyclopentyl moiety and a C₆ alkyl is a hexyl or cyclohexyl moiety and a C₇ alkyl is a heptyl or cycloheptyl, which also applies to higher ranking alkyls as generally understood by a person skilled in the art.

The term alkyl substituted C₆-aryl in the context of the present specification relates to a cyclic aromatic C₆ hydrocarbon. The C₆-aryl may be further substituted with in one or several carbon atoms by groups selected from saturated or unsaturated hydrocarbons (alkyl in their broadest sense). Examples of include but are not limited to phenyl, benzyl, xylylenyl and toluenyl.

### DETAILED DESCRIPTION OF THE INVENTION

A first aspect of the invention relates to a compound according to formula (I)

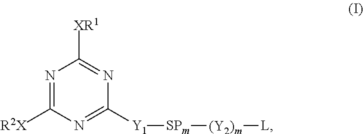

wherein,
X is selected from the group consisting of —O—(CH₂)ₙ—, wherein n is 0 to 4,
R¹ and R² are independently selected from the group consisting of C₁₁-C₂₅ alkyl, SP is a spacer, particularly selected from the group consisting of saturated C₄-C₈ alkyl, unsaturated C₄-C₈ alkyl and substituted or unsubstituted C₆-aromatic compound, in particular the substituted C₆-aromatic compound is an C₆-aromatic compound,
Y is a heteroatom, particularly selected from the group consisting of —O— and —NH—,
L is a linker for peptide synthesis, and
m is 0 or 1, particularly m is 1.

The compound is used as a tag to perform peptide elongation in greener solvents compared to SPPS. Furthermore, the new tags allow a simple work-up of the coupling reactions by simple phase separation. The resulting tag conjugated peptide remains in the organic phase and can be used directly for the next coupling step without any further purification step. The synthesis of the desired tag is established from readily available and cheap starting materials, providing an affordable molecule for LPPS.

In certain embodiments, n is 0 or 1.

In certain embodiments, n is 0.

In certain embodiments, R¹ and R² are independently from each other a branched or substituted C₁₁-C₂₅ alkyl.

In certain embodiments, R¹ and R² are independently selected from the group consisting of C₁₃-C₂₅ alkyl.

In certain embodiments R¹ is selected from the group consisting of C₁₇-C₂₅ alkyl.

In certain embodiments, R¹ is selected from the group consisting of C₂₁-C₂₅ alkyl.

In certain embodiments, R² is selected from the group consisting of C₁₇-C₂₅ alkyl.

In certain embodiments, R² is selected from the group consisting of C₂₁-C₂₅ alkyl.

In certain embodiments, R¹ and R² are a branched or substituted C₁₁-C₂₅ alkyl.

In certain embodiments, R¹ and R² are selected from the group consisting of C₁₃-C₂₅ alkyl.

In certain embodiments, R¹ and R² are selected from the group consisting of C₁₇-C₂₅ alkyl.

In certain embodiments, R¹ and R² are selected from the group consisting of C₂₁-C₂₅ alkyl.

In certain embodiments, Y₁ is —NH— and Y₂ is —NH— or —O.

In certain embodiments, Y₁ is —NH— and Y₂ is —NH—.

In certain embodiments, SP is selected from the group consisting of saturated C₄-C₈ alkyl, unsaturated C₄-C₈ alkyl and substituted C₆-aromatic compound.

In certain embodiments, SP is selected from the group consisting of saturated C₄-C₈ alkyl and an alkyl substituted C₆-aromatic compound.

In certain embodiments, SP is selected from the group consisting of saturated C₄-C₈ alkyl.

In certain embodiments, SP is selected from the group consisting of saturated C₅-C₇ alkyl and an alkyl substituted C₆-aromatic compound.

In certain embodiments, SP is selected from the group consisting of saturated C₅-C₇ alkyl.

In certain embodiments, the linker L selected from the group consisting of Rink, Sieber, Trityl, Ramage, Wang, Sasrin, HMPB, 2-chlorotrityl, MBHA, PAL, HMBA and HMPA.

In certain embodiments, the linker L is selected from the group consisting of Ramage, Rink, HMPA, PAL and HMBA. In certain embodiments, the linker L selected from the group consisting of Rink, Sieber, Trityl, Ramage, Wang, Sasrin, HMPB, 2-chlorotrityl and MBHA. In certain embodiments, the linker L is selected from the group consisting of Rink, Sieber and Trityl.

The tag can be used in peptide synthesis which in comparison to conventional Solid Phase Peptide Synthesis is performed in greener solvents and less amount of solvent making this approach a more environmentally friendly and cost-effective approach to synthesise peptides.

Furthermore, the use of the tag in peptide synthesis does not require additional purification steps which results higher peptide yields compared to conventional peptide synthesis.

The invention is further illustrated by the following examples, from which further embodiments and advantages can be drawn. These examples are meant to illustrate the invention but not to limit its scope.

### EXAMPLES

### Example 1: Synthesis of the Tags Preparation of 2-chloro-4,6-bis(heptadecan-9-yloxy)-1,3,5-triazine

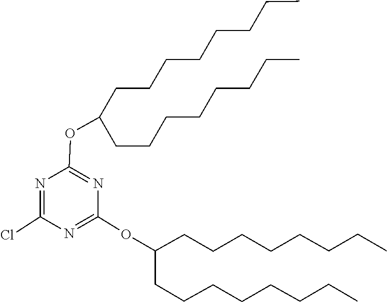 [SMILES: CCCCCCCCC(CCCCCCCC)Oc1nc(Cl)nc(OC(CCCCCCCC)CCCCCCCC)n1]

Under nitrogen atmosphere, heptadecan-9-ol (48.68 g, 3.5 eq., 189.80 mmol) was dissolved in dry, stabilised tetrahydrofuran (500 mL) and sodium hydride (11.00 g, 60% Wt, 5.0 eq., 271.10 mmol) was added in portions at 4° C. for 10 min. After complete addition the reaction was allowed to warm to room temperature and meanwhile was stirred for 1 h. Then the 2,4,6-trichloro-1,3,5-triazine (10.00 g, 1.0 eq., 54.23 mmol) was added in portions for 10 min and stirred at 40° C. for 40 h.

The reaction was quenched in an ice-bath with water (100 mL) for 15 min and was diluted with ethyl acetate (200 mL). The pH was adjusted to pH 5 by adding 1 M HCl. The mixture was transferred to a separating funnel and was extracted. The combined organic phases were washed with brine (100 mL) and dried over MgSO₄. The solvent was removed under reduced pressure, obtaining the crude (16.24 g, 26.0 mmol, 48.0%) as a white wax-like powder.

The crude was further purified by column chromatography (1000 g, SiO₂; EtOAc/n-heptane, 1:50), obtaining the product as a light-yellow oil.

MS (ESI, positive ion): m/z=624.53 [M+H]⁺

### Preparation of N1-(4,6-bis(heptadecan-9-yloxy)-1,3,5-triazin-2-yl)hexane-1,6-diamine

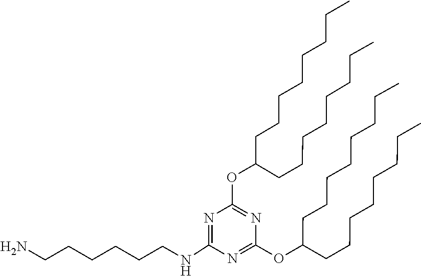 [SMILES: CCCCCCCCC(CCCCCCCC)Oc1nc(NCCCCCCN)nc(OC(CCCCCCCC)CCCCCCCC)n1]

Hexane-1,6-diamine (10.24 g, 5.0 eq., 88.079 mmol) was dissolved in toluene (200 mL) and stirred for 1 h at room temperature. 2-chloro-4,6-bis(heptadecan-9-yloxy)-1,3,5-triazine (11.00 g, 1.0 eq., 17.616 mmol) was dissolved in toluene (100 mL) and added to the reaction. Then diisopropylethylamine (17.00 g, 23 mL, 7.6 eq., 0.13 mol) was added and the reaction stirred for 48 h at 80° C. The solvent was removed under reduced pressure. The mixture was diluted in 200 mL ethyl acetate and was transferred to a separating funnel and was extracted three times with water (100 mL) and with brine (50 mL). The solvent was removed under reduced pressure, obtaining the crude (9.85 g, 13.98 mmol, 79.4%) as a yellowish oil.

The product was used without further purification.

MS (ESI, positive ion): m/z=704.66 [M+H]⁺

### Preparation of (9H-fluoren-9-yl)methyl ((4-(2-((6-((4,6-bis(heptadecan-9-yloxy)-1,3,5-triazin-2-yl)amino)hexyl)amino)-2-oxoethoxy)phenyl)(2,4-dimethoxyphenyl)methyl)carbamate

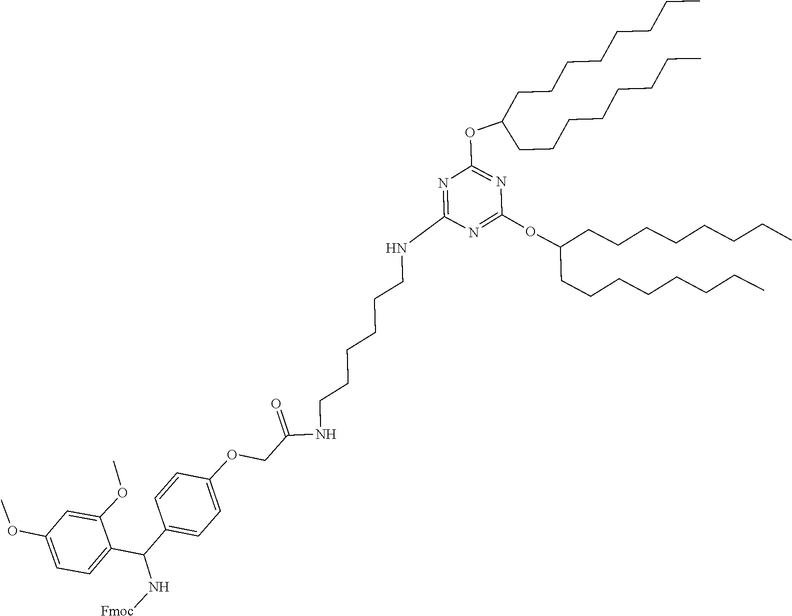

Fmoc-Rink-Linker (15.33 g, 2.0 eq., 28.40 mmol) and HOBt (4.86 g, 2.0 eq., 28.40 mmol) were dissolved in dichloromethane (250 mL) and EDC HCl (5.50 g, 99% Wt, 2.0 eq., 28.40 mmol) was added in portions over 5 min. After stirring at room temperature for 1 h, the reaction was cooled in an ice-bath (4° C. internal) and 4-methylmorpholine (2.87 g, 3.2 mL, 2.0 eq., 28.40 mmol) was added. Following, N¹-(4,6-bis(heptadecan-9-yloxy)-1,3,5-triazin-2-yl)hexane-1,6-diamine (10.00 g, 1.0 eq., 14.20 mmol) was added in portions for 5 min, keeping the internal temperature below 10° C. After complete addition the reaction mixture was allowed to warm to room temperature.

After stirring for 8 h at 40° C. the suspension was evaporated to remove the dichloromethane. The crude was diluted with ethyl acetate and washed three times with water (100 mL) and one time with brine (100 mL) yielding (9H-fluoren-9-yl)methyl ((4-(2-((6-((4,6-bis(heptadecan-9-yloxy)-1,3,5-triazin-2-yl)amino)hexyl)amino)-2-oxoethoxy)phenyl)(2,4-dimethoxyphenyl)-methyl) carbamate (12.52 g, 10.21 mmol, 71.9%).

MS (ESI, positive ion): m/z=1225.83 [M+H]⁺

¹H NMR (400 MHz, CD₂Cl₂) δ [ppm]=7.77, 7.76 (s, 2H), 7.60-7.59 (m, 2H), 7.57-7.56 (m, 2H), 7.38-7.37 (m, 2H), 7.30-7.28 (m, 2H), 7.15-7.06 (m, 2H), 6.86-6.78 (m, 2H), 6.49-6.45 (m, 1H), 6.22 (s, 1H), 6.10-5.95 (m, 2H), 5.32-5.30 (m, 2H), 5.16 (s, 2H, OCH), 4.42-4.38 (m, 2H), 3.78-3.72 (m, 6H), 3.35-3.25 (m, 2H), 2.78-2.76 (m, 2H), 1.68-1.48 (m, 12H), 1.35-1-05 (m, 54H), 0.89-0.85 (m, 12H).

¹³C-NMR (101 MHz, CD₂Cl₂): δ [ppm]=173.7, 173.4, 168.9, 168.2, 161.1, 158.6, 156.8, 156.0, 144.6, 141.7, 136.4, 134.2, 129.2, 127.5, 125.5, 121.4, 120.3, 114.8, 109.8, 104.8, 99.6, 85.8, 78.4, 77.4, 68.0, 67.9, 54.1, 53.8, 51.2, 47.8, 41.2, 40.5, 39.1, 34.5, 32.3, 30.0, 29.9, 26.9, 25.8, 23.1, 19.3, 15.8, 14.3.

### Preparation of (9H-fluoren-9-yl)methyl (2-(2-((6-((4,6-bis(heptadecan-9-yloxy)-1,3,5-triazin-2-yl)amino)hexyl)amino)-2-oxoethoxy)-10,11-dihydro-5H-dibenzo[a,d][7]annulen-5-yl)-carbamate

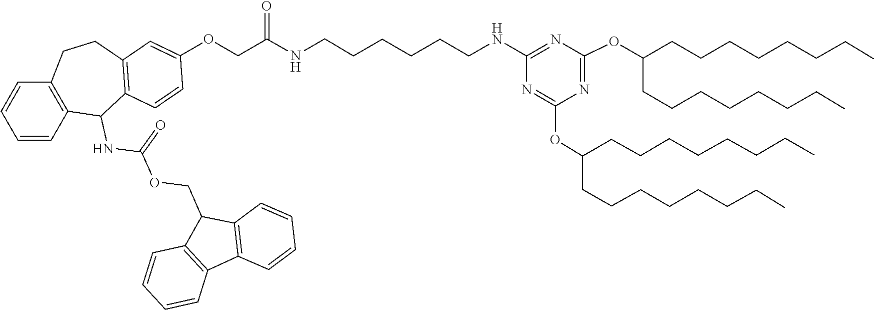 [SMILES: CCCCCCCCC(CCCCCCCC)Oc1nc(NCCCCCCNC(=O)COc2ccc3c(c2)CCc2ccccc2C3NC(=O)OCC2c3ccccc3-c3ccccc32)nc(OC(CCCCCCCC)CCCCCCCC)n1]

Fmoc-Ramage-Linker (3.95 g, 1.1 eq., 7.81 mmol) and HOBt (1.46 g, 1.2 eq., 8.52 mmol) were dissolved in DMF (40 mL), the reaction was cooled in an ice-bath (4° C. internal). 4-methylmorpholine (1.46 g, 1.2 eq., 8.52 mmol) was added at 4° C. followed by the addition of EDC HCl (2.06 g, 99% Wt, 1.5 eq., 10.7 mmol) in two portions. Following, N¹-(4,6-bis(heptadecan-9-yloxy)-1,3,5-triazin-2-yl)hexane-1,6-diamine (5.00 g, 1.0 eq., 7.10 mmol) was dissolved in DMF (31 mL) and added in portions for 3 min, keeping the internal temperature below 10° C. After complete addition the reaction mixture was allowed to warm to room temperature.

After stirring for 3 h at 22° C. the crude was diluted with 71 mL acetonitrile and cooled to 4° C. 71 mL water was added slowly and a precipitate formed, another 71 mL water was added at 22° C. and the precipitate was collected via filtration through a P3 glass frit. The precipitate was washed with 70/30 water/acetonitrile (100 mL) and 90/10 water/acetonitrile (100 mL). Afterwards, the precipitate was dissolved in 200 mL 2-methyltetrahydrofuran, washed with brine (100 mL) and dried over MgSO₄. The crude material was purified by MPLC (4 runs on 24 g SiO₂ column) using a gradient of DCM to DCM/EtOAc 80:20 yielding (9H-fluoren-9-yl)methyl-(2-(2-((6-((4,6-bis(heptadecan-9-yloxy)-1,3,5-triazin-2-yl)amino)hexyl)amino)-2-oxoethoxy)-10,11-dihydro-5H-dibenzo[a,d][7]annulen-5-yl)carbamate (5.39 g, 4.52 mmol, 63.7%).

MS (ESI, positive ion): m/z=1191.87 [M+H]⁺

¹H NMR (400 MHz, CD₂Cl₂) δ [ppm]=7.78 (d, J=7.5 Hz, 2H), 7.54 (d, J=7.5 Hz, 2H), 7.40 (t, J=7.5 Hz, 2H), 7.29 (td, J=7.5, 1.2 Hz, 3H), 7.19 (dt, J=16.7, 4.9 Hz, 3H), 6.74 (d, J=2.3 Hz, 2H), 6.52 (t, J=6.0 Hz, 1H), 5.20-5.04 (m, 2H), 4.43 (s, 4H), 4.20 (t, J=6.5 Hz, 1H), 3.32 (dp, J=19.9, 6.9 Hz, 6H), 3.04 (q, J=12.4 Hz, 2H), 1.58 (ddt, J=25.5, 20.7, 7.5 Hz, 16H), 1.27 (d, J=7.2 Hz, 54H), 0.92-0.84 (m, 12H).

¹³C-NMR (101 MHz, CD₂Cl₂): δ [ppm]=172.8, 169.0, 168.1, 157.5, 155.4, 144.5, 141.8, 141.4, 139.0, 132.7, 131.3, 130.8, 129.5, 128.4, 128.1, 127.5, 126.8, 125.4, 120.4, 116.9, 112.6, 77.5, 77.1, 68.0, 66.9, 60.1, 47.8, 41.2, 39.2, 34.6, 34.5, 33.3, 32.8, 32.3, 30.1, 30.0, 30.0, 29.9, 29.7, 26.9, 26.9, 25.9, 25.8, 23.1, 14.3.

### Preparation of 2,4-bis((2-butyloctyl)oxy)-6-chloro-1,3,5-triazine

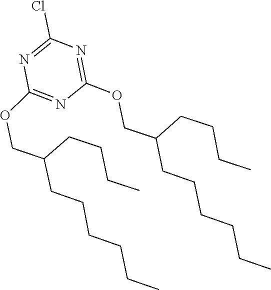 [SMILES: CCCCCCC(CCCC)COc1nc(Cl)nc(OCC(CCCC)CCCCCC)n1]

Under a nitrogen atmosphere 2-butyloctan-1-ol (2.02 g, 2.4 mL, 2.0 eq., 10.8 mmol) was dissolved in 2-methyltetrahydrofuran (20 mL) at room temperature. The reaction mixture was cooled to 0° C. and sodium hydride (2.17 g, 60% Wt, 10.0 eq., 54.20 mmol) was added in portions. After complete addition the reaction mixture was refluxed for 18 h. After cooling to −10° C. the activated alcohol was added dropwise in three portions to a cooled solution (−10° C.) of 2,4,6-trichloro-1,3,5-triazine (1.00 g, 1.0 Eq., 5.42 mmol) dissolved in 2-methyltetrahydrofuran (10 mL). The reaction mixture was stirred and allowed to warm to room temperature for 4 h before it was quenched with H₂O, transferred to a separating funnel and washed with brine (50 mL). The organic phase was dried over MgSO₄ to yield 2,4-bis((2-butyloctyl)oxy)-6-chloro-1,3,5-triazine. The product was used without further purification.

MS (ESI, positive ion): m/z=484.33 [M+H]⁺

### Preparation of N′-(4,6-bis((2-butyloctyl)oxy)-1,3,5-triazin-2-yl)hexane-1,6-diamine

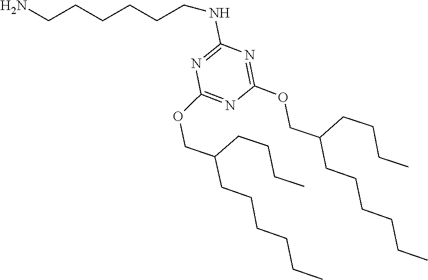 [SMILES: CCCCCCC(CCCC)COc1nc(NCCCCCCN)nc(OCC(CCCC)CCCCCC)n1]

Hexane-1,6-diamine (6.31 g, 10.0 eq., 54.30 mmol) was dissolved in 2-methyltetrahydrofuran (30 mL) and stirred for 10 min at elevated temperatures. The diamine solution was heated to 70° C. and a solution of 2,4-bis((2-butyloctyl)oxy)-6-chloro-1,3,5-triazine (2.63 g, 1.0 Eq., 5.43 mmol) in 2-methyltetrahydrofuran (20 mL) was added dropwise. After a complete addition the reaction was stirred for ca. 1.5 h. Afterwards, the reaction was cooled to room temperature and the precipitate was filtered off (G3-Fritte). The remaining solution was diluted with ethyl acetate (600 mL). The organic layer was washed four times with water (4×250 mL), one time with 0.5 M HCl (250 mL) and one time with brine (250 mL). The combined organic phases were dried over MgSO₄ and purified by column chromatography (100 g, SiO₂; DCM:NH₃ (7N) in MeOH 90:10) to yield N1-(4,6-bis((2-butyloctyl)oxy)-1,3,5-triazin-2-yl)hexane-1,6-diamine (1.10 g, 1.96 mmol, 36.0% over 2 steps).

MS (ESI, positive ion): m/z=564.52 [M+H]⁺

### Preparation of (9H-fluoren-9-yl)methyl ((4-(2-((6-((4,6-bis((2-butyloctyl)oxy)-1,3,5-triazin-2-yl)amino)hexyl)amino)-2-oxoethoxy)phenyl)(2,4-dimethoxyphenyl)methyl)carbamate

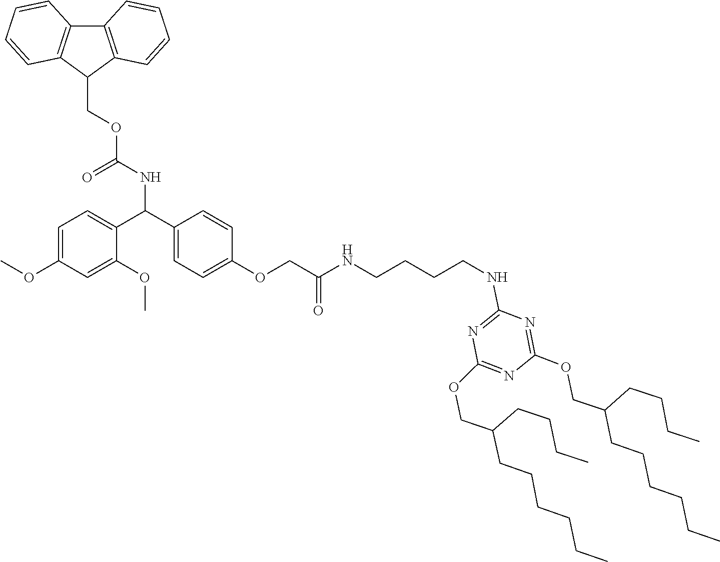 [SMILES: CCCCCCC(CCCC)COc1nc(NCCCCNC(=O)COc2ccc(C(NC(=O)OCC3c4ccccc4-c4ccccc43)c3ccc(OC)cc3OC)cc2)nc(OCC(CCCC)CCCCCC)n1]

To a solution of 2-(4-(((((9H-fluoren-9-yl)methoxy)carbonyl)amino)(2,4-dimethoxyphenyl)-methyl)phenoxy)acetic acid (526 mg, 1.1 eq., 975 μmol) in DMF (5 mL), HOBt Dihydrat (182 mg, 1.2 eq., 1.06 mmol) was added and the mixture was cooled to 4° C. EDC HCl (258 mg, 99% Wt, 1.5 eq., 1.33 mmol) was added followed by 4-methylmorpholine (359 mg, 0.39 mL, 4.0 eq., 3.55 mmol) and the mixture was stirred at 4° C. for 5 min. Afterwards, a solution of N¹-(4,6-bis((2-butyloctyl)oxy)-1,3,5-triazin-2-yl)hexane-1,6-diamine (526 mg, 1.1 eq., 975 μmol) in DMF (5 mL) was added. The mixture was stirred at 4° C. for another 5 min, then allowed to warm and stirred at room temperature for 1 h. The mixture was diluted with acetonitrile (10 mL) and added dropwise to 40 mL dest. H₂O at 4° C. The resulting mixture was diluted with dichloromethane (150 mL) as well as 5w % LiCl (50 mL) and the mixture was transferred to a separating funnel. The resulting emulsion could be cleared by addition of 20 mL ethanol. The phases were separated and the aqueous phase was extracted twice with dichloromethane (50 mL). The combined organic phases were washed with 5 w % LiCl and brine, then dried over MgSO₄ and purified by MPLC (12 g SiO₂ column) using a gradient of DCM to DCM/EtOAc 80/20 to yield (9H-fluoren-9-yl)methyl ((4-(2-((6-((4,6-bis((2-butyloctyl)oxy)-1,3,5-triazin-2-yl)amino)hexyl)amino)-2-oxoethoxy)phenyl)(2,4-dimethoxyphenyl)methyl)carbamate (480 mg, 442 μmol, 49.9%).

MS (ESI, positive ion): m/z=1085.71 [M+H]⁺

¹H NMR (400 MHz, CD₂Cl₂) δ [ppm]=7.78 (d, J=7.6 Hz, 2H), 7.60 (s, 1H), 7.40 (td, J=7.5, 3.1 Hz, 2H), 7.30 (s, 2H), 7.15 (d, J=8.4 Hz, 2H), 7.09 (d, J=8.2 Hz, 1H), 6.89-6.80 (m, 2H), 6.55 (t, J=5.9 Hz, 1H), 6.51-6.45 (m, 2H), 5.98 (d, J=8.5 Hz, 1H), 4.43 (s, 4H), 4.23 (dd, J=8.7, 5.9 Hz, 3H), 4.15 (d, J=5.8 Hz, 2H), 3.80 (s, 3H), 3.74 (s, 3H), 3.38 (td, J=7.1, 5.9 Hz, 2H), 3.30 (q, J=6.8 Hz, 2H), 1.60-1.47 (m, 4H), 1.37-1.24 (m, 38H), 0.96-0.84 (m, 12H).

¹³C-NMR (101 MHz, CD₂Cl₂): δ [ppm]=172.9, 168.8, 168.2, 161.2, 158.4, 156.8, 144.5, 141.8, 136.4, 129.7, 128.4, 128.0, 127.4, 125.5, 125.5, 120.3, 114.9, 104.8, 99.7, 70.5, 70.3, 68.1, 55.9, 55.8, 47.9, 41.3, 39.2, 38.1, 32.3, 31.7, 31.3, 30.1, 30.0, 29.9, 29.5, 27.2, 26.9, 26.9, 23.5, 23.1, 14.3.

### Preparation of N-(3-((12-azaneyl)methyl)benzyl)-4,6-bis(heptadecan-9-yloxy)-1,3,5-triazin-2-amine

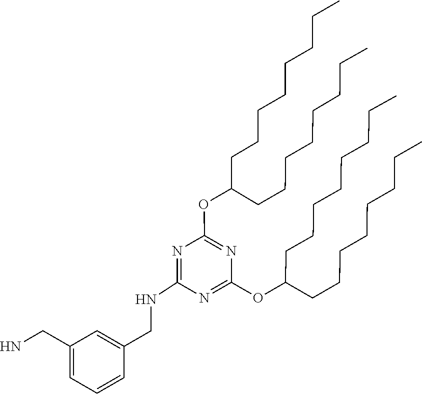 [SMILES: CCCCCCCCC(CCCCCCCC)Oc1nc(NCc2cccc(CN)c2)nc(OC(CCCCCCCC)CCCCCCCC)n1]

1,3-phenylenedimethanamine (7.39 g, 7.16 mL, 10.0 eq., 54.30 mmol) was dissolved in 2-methyltetrahydrofuran (30 mL) and stirred for 10 min at elevated temperatures. A solution of 2-chloro-4,6-bis(heptadecan-9-yloxy)-1,3,5-triazine (3.39 g, 1.0 eq., 5.43 mmol) in 2-methyltetrahydrofuran (35 mL) was added dropwise. After complete addition the reaction was stirred for ca. 1 h and afterwards cooled to room temperature and the precipitate was filtered off (G3-Fritte). The remaining solution was diluted with ethyl acetate (600 mL). The organic layer was washed four times with water (4×250 mL), one time with 0.5 M HCl (250 mL) and one time with brine (250 mL), dried over MgSO₄ and evaporated. The crude was further purified by column chromatography (100 g, SiO₂; DCM/MeOH, 95:5-91:10, followed by DCM:NH₃ (7N) in MeOH 90:10), obtaining the product as a light-yellow oil (1.00 g, 1.39 mmol, 25.5%).

MS (ESI, positive ion): m/z=724.67 [M+H]⁺

### Preparation of 4-(((triisopropylsilyl)oxy)methyl)benzoic acid

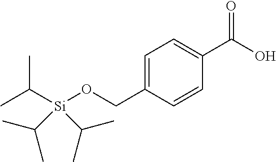 [SMILES: CC(C)[Si](OCc1ccc(C(=O)O)cc1)(C(C)C)C(C)C]

To a suspension of 4-(hydroxymethyl)benzoic acid (0.85 g, 1.0 eq., 5.60 mmol) in dichloromethane (30 mL), imidazole (1.10 g, 3.0 eq., 17.00 mmol) was added, followed by dropwise addition of triisopropylsilyltriflate (3.50 g, 3.10 mL, 2.0 eq., 11.00 mmol) at 4° C. The mixture was allowed to warm to room temperature and then heated to reflux for 3 h. After this time, the solvent was removed under reduced pressure and the residue was dissolved in THF/H2O (1:1; 30 mL). Afterwards, potassium carbonate (0.77 g, 1.0 eq., 5.60 mmol) was added and the mixture was heated to −50° C. for 1 h. The reaction mixture was transferred to a separating funnel and acidified to pH ~3.5 by addition of 5w % citric acid. Ethyl acetate was added and the phases were separated. The aqueous phase was extracted with Ethyl acetate and the combined organic extracts were washed two times with H₂O (2×30 mL) and one time with brine (30 mL). The crude mixture was dried over MgSO₄ and the solvent removed under reduced pressure. An analytically pure sample was obtained by column chromatography, but the bulk material was used in the next step without further purification.

¹H NMR (400 MHz, CD₂Cl₂) δ [ppm]=8.14-8.00 (m, 2H), 7.50 (m, 2H), 4.93 (s, 2H), 1.13-1.08 (m, 21H).

### Preparation of N-(3-(((4,6-bis(heptadecan-9-yloxy)-1,3,5-triazin-2-yl)amino)methyl)benzyl)-4-(((triisopropylsilyl)oxy)methyl)benzamide

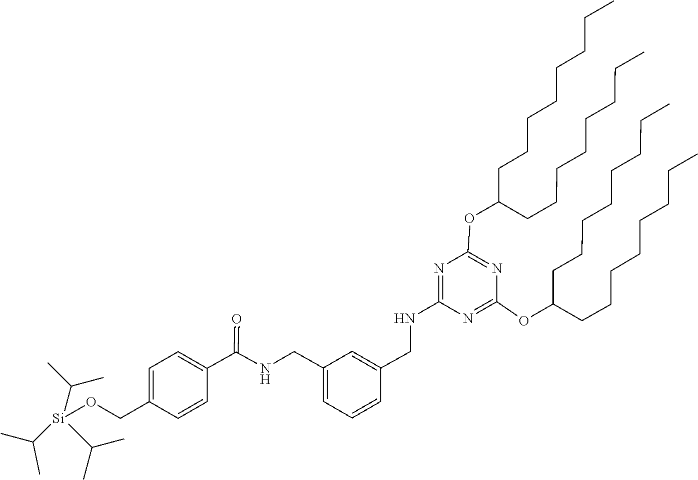

To a solution of N-(3-(aminomethyl)benzyl)-4,6-bis(heptadecan-9-yloxy)-1,3,5-triazin-2-amine (181 mg, 1.0 eq., 250 μmol) in dichloromethane (10 mL), 4-(((triisopropylsilyl)oxy)methyl)-benzoic acid (92.5 mg, 1.2 eq., 300 μmol) and 1H-benzo[d][1,2,3]triazol-1-ol hydrate (49.8 mg, 1.3 eq., 325 μmol) were added followed by 1-(3-Dimethylaminopropyl)-3-ethylcarbodiimide Hydrochloride (71.9 mg, 1.50 eq., 375 μmol) as well as 4-methylmorpholine (0.13 g, 0.14 mL, 5.1 eq., 1.30 mmol) and the mixture was stirred at room temperature for 2.5 h. After this time the reaction mixture was transferred into a separating funnel and washed with 1 M KHSO₄, sat. NaHCO₃ and brine. The organic phase was dried over MgSO₄ and the solvent removed under reduced pressure. The crude was further purified by MPLC (4 g SiO₂ column) using a gradient of DCM to DCM/EtOAc 80/20, obtaining the product as a light-yellow oil (66 mg, 250 μmol, 26%).

MS (ESI, positive ion): m/z=1014.77 [M+H]⁺

¹H NMR (400 MHz, CD₂Cl₂) δ [ppm]=8.08-8.01 (m, 2H), 7.79-7.72 (m, 2H), 7.47 (dd, J=7.9, 0.8 Hz, 2H), 7.45-7.40 (m, 2H), 4.92 (s, 2H), 4.88 (d, J=0.9 Hz, 2H), 4.62 (dd, J=5.9, 4.4 Hz, 4H), 1.27 (d, J=6.5 Hz, 42H), 1.13-1.04 (m, 35H), 0.91-0.82 (m, 12H).

¹³C-NMR (101 MHz, CD₂Cl₂): δ [ppm]=172.7, 172.1, 170.4, 168.7, 167.4, 148.2, 146.0, 139.7, 139.5, 133.4, 130.3, 129.4, 128.7, 127.3, 127.2, 127.1, 126.9, 126.2, 126.0, 77.9, 77.6, 65.1, 65.1, 45.0, 44.2, 34.5, 34.4, 32.3, 30.1, 30.0, 29.7, 25.8, 25.7, 23.1, 18.2, 14.3, 12.5.

### Preparation of 2-chloro-4,6-bis((16-methylheptadecyl)oxy)-1,3,5-triazine

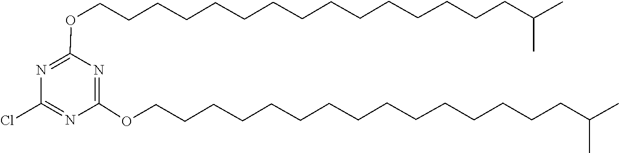 [SMILES: CC(C)CCCCCCCCCCCCCCCOc1nc(Cl)nc(OCCCCCCCCCCCCCCCC(C)C)n1]

Under a nitrogen atmosphere 16-Methyl-1-heptadecanol (2.93 g, 2.0 eq., 10.8 mmol) was dissolved in 2-MeTHF (20 mL), cooled to 0° C. and sodium hydride (2.17 g, 60% Wt, 10.0 eq., 54.2 mmol) was added in portions. After complete addition the reaction mixture was refluxed for 18 h. After cooling to −10° C. the activated alcohol was added dropwise over 15 min to a cooled solution (−10° C.) of 2,4,6-trichloro-1,3,5-triazine (1.00 g, 1.0 eq., 5.42 mmol) dissolved in 2-MeTHF (10 mL). The reaction mixture was stirred and allowed to warm to room temperature for 1 h before it was quenched using cold water and transferred to a separating funnel, washed one time with brine (50 mL). The organic phase was dried over MgSO₄ to yield 2-chloro-4,6-bis((16-methylheptadecyl)oxy)-1,3,5-triazine.

MS (ESI, positive ion): m/z=652.62 [M+H]⁺

### Preparation of N-(4-(λ²-azaneyl)butyl)-4,6-bis((16-methylheptadecyl)oxy)-1,3,5-triazin-2-amine

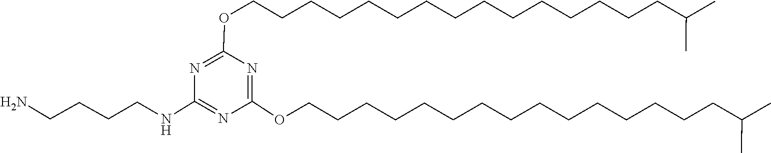 [SMILES: CC(C)CCCCCCCCCCCCCCCOc1nc(NCCCCN)nc(OCCCCCCCCCCCCCCCC(C)C)n1]

Butane-1,4-diamine (4.78 g, 10.0 eq., 54.3 mmol) was dissolved in 2-MeTHF (30 mL) and stirred for 10 min at elevated temperatures. A solution of 2-chloro-4,6-bis((16-methylheptadecyl)oxy)-1,3,5-triazine (3.54 g, 1.0 eq., 5.43 mmol) in 40 mL 2-MeTHF was added dropwise. After the complete additions, the reaction was further stirred for 1.5 h. The reaction was then cooled to room temperature and the precipitate was filtered off (G3-Fritte). The remaining solution was diluted with 600 mL of EE. The organic layer was washed with water (4×250 mL) and 0.5 M HCl (250 mL). The organic phase was washed with brine (250 mL). The combined organic phases were dried over MgSO₄ and evaporated. The crude was further purified by MPLC (24 g SiO₂ column) using a gradient using DCM/MeOH obtaining the product as a colourless wax (1.35 g, 1.92 mmol, 35.3% over 2 steps).

MS (ESI, positive ion): m/z=704.69 [M+H]⁺

### Preparation of (9H-fluoren-9-yl)methyl (4-((5-((4-((4,6-bis((16-methylheptadecyl)oxy)-1,3,5-triazin-2-yl)amino)butyl)amino)-5-oxopentyl)oxy)-2,6-dimethoxybenzyl)carbamate

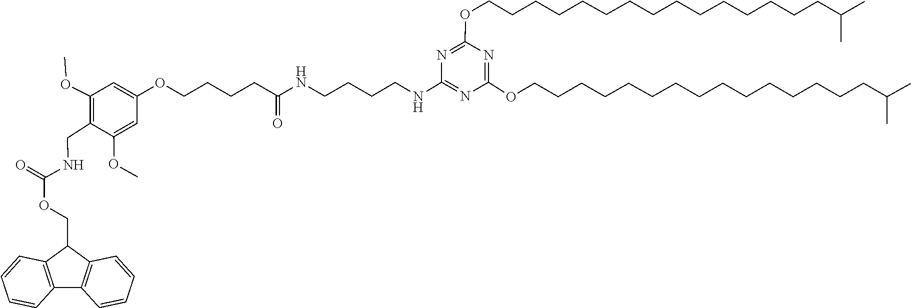 [SMILES: COc1cc(OCCCCC(=O)NCCCCNc2nc(OCCCCCCCCCCCCCCCC(C)C)nc(OCCCCCCCCCCCCCCCC(C)C)n2)cc(OC)c1CNC(=O)OCC1c2ccccc2-c2ccccc21]

Fmoc-PAL (359 mg, 1.0 eq., 710 μmol) was dissolved in DMF (4 mL). Afterwards, HOBt Dihydrat (146 mg, 1.2 eq., 852 μmol) was added and the mixture was cooled to 4° C. 4-methylmorpholine (287 mg, 0.32 mL, 4.0 eq., 2.84 mmol) was added at 4° C. followed by addition of EDC HCl (206 mg, 99% Wt, 1.5 eq., 1.07 mmol) in two portions. N-(4-(λ²-azaneyl)butyl)-4,6-bis((16-methylheptadecyl)oxy)-1,3,5-triazin-2-amine (500 mg, 1.0 eq., 710 μmol) was dissolved in DMF (3 mL) and added to the mixture. The reaction mixture was stirred at 4° C. for another 5 min, warmed to room temperature and stirred at this temperature for 1.

After a total stirring time of 3 h, the mixture was diluted with acetonitrile (8 mL) and cooled to 4° C. 20 mL dest. H₂O were added slowly at 4° C. and an oily precipitate formed. The precipitate was collected by filtration through a P3 glass fritt, washed with 100 mL H₂O, dissolved in dichloromethane (100 mL) and washed with brine (100 mL). The organic phase was dried over MgSO₄ and the solvent was removed under reduced pressure. The crude was further purified by MPLC (12 g SiO₂ column) using a gradient of DCM to DCM/EtOAc 80/20, obtaining the product as a light-yellow oil (702 mg, 589 μmol, 83.0%).

MS (ESI, positive ion): m/z=1191.94 [M+H]⁺

¹H NMR (400 MHz, CD₂Cl₂) δ [ppm]=7.77 (d, J=7.6 Hz, 2H), 7.60 (d, J=7.5 Hz, 2H), 7.39 (t, J=7.4 Hz, 2H), 7.30 (t, J=7.5 Hz, 2H), 6.14 (s, 2H), 5.81-5.62 (m, 2H), 5.17 (s, 1H), 4.34 (d, J=7.1 Hz, 4H), 4.27-4.13 (m, 4H), 4.06-3.93 (m, 2H), 3.81 (s, 6H), 3.56-3.37 (m, 2H), 3.34-3.19 (m, 2H), 2.32-2.12 (m, 2H), 2.01 (s, 1H), 1.89-1.74 (m, 6H), 1.66-1.50 (m, 4H), 1.48-1.19 (m, 56H), 0.98-0.74 (m, 12H).

¹³C-NMR (101 MHz, CD₂Cl₂): δ [ppm]=172.7, 168.8, 160.9, 159.7, 156.5, 144.8, 141.7, 128.0, 127.4, 125.5, 120.3, 107.4, 91.6, 70.6, 68.2, 66.8, 56.2, 54.4, 54.3, 54.1, 54.0, 53.8, 53.8, 53.6, 53.3, 47.8, 41.0, 39.5, 38.1, 36.5, 34.2, 32.4, 32.4, 32.3, 31.6, 30.5, 30.1, 30.1, 30.0, 29.9, 29.8, 29.8, 29.8, 29.3, 27.4, 27.4, 27.2, 23.1, 22.8, 14.3.

### Preparation of 2-chloro-4,6-bis((2-decyltetradecyl)oxy)-1,3,5-triazine

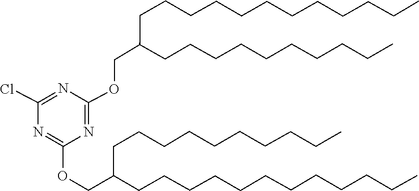 [SMILES: CCCCCCCCCCCCC(CCCCCCCCCC)COc1nc(Cl)nc(OCC(CCCCCCCCCC)CCCCCCCCCCCC)n1]

Under nitrogen atmosphere 2-decyltetradecanol (3.85 g, 4.57 mL, 2.0 eq., 10.80 mmol) was dissolved in 2-methyltetrahydrofuran (20 mL), cooled to 0° C. and sodium hydride (2.17 g, 60% Wt, 10.0 eq., 54.20 mmol) was added in portions. After complete addition the reaction mixture was refluxed for 18 h. After cooling to −10° C. the activated alcohol was added dropwise to a cooled solution (−10° C.) of 2,4,6-trichloro-1,3,5-triazine (1.00 g, 1.0 Eq., 5.42 mmol) dissolved in 2-methyltetrahydrofuran (10 mL). The reaction mixture was stirred and allowed to warm to room temperature for 1 h before it was quenched with H₂O and washed with brine (40 mL). The organic phase was dried over MgSO₄ and the solvent removed under vacuum to yield 2-chloro-4,6-bis((2-decyltetradecyl)oxy)-1,3,5-triazine.

MS (ESI, positive ion): m/z=820.75 [M+H]⁺

### Preparation of N′-(4,6-bis((2-decyltetradecyl)oxy)-1,3,5-triazin-2-yl)octane-1,8-diamine

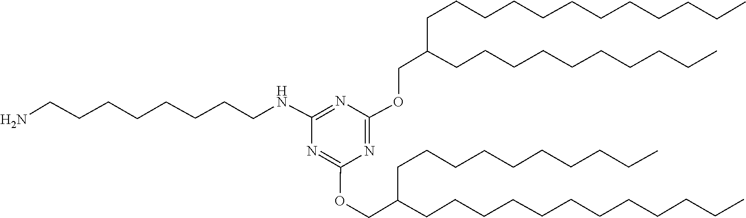 [SMILES: CCCCCCCCCCCCC(CCCCCCCCCC)COc1nc(NCCCCCCCCN)nc(OCC(CCCCCCCCCC)CCCCCCCCCCCC)n1]

Octane-1,8-diamine (7.82 g, 10.0 eq., 54.2 mmol) was dissolved in 2-methyltetrahydrofuran (30 mL) and stirred for 10 min at elevated temperatures. The Diamine solution was heated to 70° C. and a solution of 2-chloro-4,6-bis((2-decyltetradecyl)oxy)-1,3,5-triazine (4.45 g, 1.0 eq., 5.42 mmol) in 2-methyltetrahydrofuran (40 mL) was added dropwise. The reaction was stirred for ca. 1.5 h, before it was cooled to room temperature and the precipitate was filtered off (G3-Fritte). The remaining solution was diluted with ethyl acetate (300 mL). The organic layer was washed four times with water (4×250 mL), one time with 0.5 M HCl (250 mL) and one time with brine (250 mL). The combined organic phases were dried over MgSO₄ and evaporated. The crude was further purified by MPLC (24 g SiO₂ column) using a gradient using DCM/MeOH, obtaining N¹-(4,6-bis((2-decyltetradecyl)oxy)-1,3,5-triazin-2-yl)octane-1,8-diamine as a colourless wax (650 mg, 700 μmol, 12.9% over 2 steps).

MS (ESI, positive ion): m/z=928.88 [M+H]⁺

### Preparation of 2-(4-(((triisopropylsilyl)oxy)methyl)phenoxy)acetic acid (HMPA-TIPS)

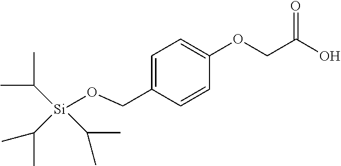 [SMILES: CC(C)[Si](OCc1ccc(OCC(=O)O)cc1)(C(C)C)C(C)C]

To a suspension of 2-(4-(hydroxymethyl)phenoxy)acetic acid (0.50 g, 1.0 eq., 2.70 mmol) in dichloromethane (20 mL), imidazole (0.56 g, 3.0 eq., 8.20 mmol) was added, followed by dropwise addition of triisopropylsilyltriflate (2.10 g, 1.80 mL, 2.5 eq., 6.90 mmol) at 4° C. The mixture was allowed to warm to room temperature and then stirred for 3 d. After this time, the suspension was heated to reflux for 2 h and the solvent was removed under reduced pressure. The residue was dissolved in THF/H₂O (1:1; 20 mL). Afterwards, potassium carbonate (0.38 g, 1.0 eq., 2.70 mmol) was added and the mixture was heated to 50° C. for 1 h. The reaction mixture was transferred to a separating funnel and acidified to pH ~3.5 by addition of 5w % citric acid. Ethyl acetate (15 mL) was added and the phases were separated. The aqueous phase was extracted with ethyl acetate and the combined organic phases were washed two times with H₂O (2×20 mL) and one time with brine (20 mL). The crude mixture was dried over MgSO₄ and the solvent removed under reduced pressure to yield 2-(4-(((triisopropylsilyl)oxy)methyl)phenoxy)acetic acid which was used without further purification.

¹H NMR (400 MHz, CD₂Cl₂) δ [ppm]=7.35-7.26 (m, 2H), 6.93-6.85 (m, 2H), 4.78 (s, 2H), 4.67 (s, 2H), 1.25-1.00 (m, 21H).

### Preparation of N-(8-((4,6-bis((2-decyltetradecyl)oxy)-1,3,5-triazin-2-yl)amino) octyl)-2-(4-(((triisopropylsilyl)oxy)methyl)phenoxy)acetamide

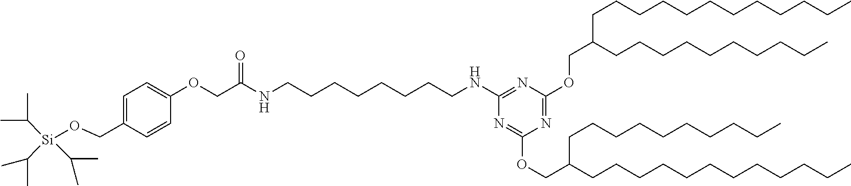 [SMILES: CCCCCCCCCCCCC(CCCCCCCCCC)COc1nc(NCCCCCCCCNC(=O)COc2ccc(CO[Si](C(C)C)(C(C)C)C(C)C)cc2)nc(OCC(CCCCCCCCCC)CCCCCCCCCCCC)n1]

HMPA-TIPS (492 mg, 3.0 eq., 1.45 mmol) was dissolved in dichloromethane (8 mL), HOBt Dihydrat (99.5 mg, 1.2 eq., 582 μmol) was added and the mixture was cooled to 4° C. 4-methylmorpholine (196 mg, 0.22 mL, 4.0 eq., 1.94 mmol) was added at 4° C. followed by addition of EDC HCl (141 mg, 99% Wt, 1.5 eq., 727 μmol) in two portions. N¹-(4,6-bis((2-decyltetradecyl)oxy)-1,3,5-triazin-2-yl)octane-1,8-diamine (450 mg, 1.0 eq., 485 μmol) was dissolved in dichloromethane (4 mL) and added to the mixture over 3 min at 4° C. The mixture was stirred for another 5 min, warmed to room temperature and stirred at this temperature for 1.5 h. The mixture was diluted with acetonitrile (3 mL) and dest. H₂O (20 mL) were added. The resulting byphasic mixture transferred to a separating funnel. 100 mL brine were added and the aqueous phase was separated. The organic phase was dried over MgSO₄ and the solvent was removed under reduced pressure. The crude was further purified by MPLC (12 g SiO₂ column) using a gradient using DCM/EE, obtaining the product as a light-yellow oil (217 mg, 174 μmol, 35.8%).

MS (ESI, positive ion): m/z=1249.15 [M+H]⁺

¹H NMR (400 MHz, CD₂Cl₂) δ [ppm]=7.31 (d, J=8.9 Hz, 2H), 6.91 (d, J=8.7 Hz, 2H), 6.56 (s, 2H), 4.77 (s, 2H), 4.45 (s, 2H), 4.18 (dd, J=25.6, 5.9 Hz, 4H), 3.43-3.36 (m, 2H), 3.34-3.26 (m, 2H), 1.87-1.68 (m, 2H), 1.56 (d, J=6.5 Hz, 4H), 1.44-1.30 (m, 28H), 1.27 (s, 60H), 1.10 (s, 12H), 1.09 (s, 6H), 1.05 (s, 3H), 0.96-0.81 (m, 12H).

¹³C-NMR (101 MHz, CD₂Cl₂): δ [ppm]=172.9, 172.4, 168.8, 168.2, 156.9, 135.9, 127.9, 114.9, 70.5, 70.3, 68.2, 65.1, 54.4, 54.3, 54.1, 54.0, 53.8, 53.8, 53.6, 53.3, 41.4, 39.4, 38.1, 38.1, 32.4, 31.7, 30.5, 30.2, 30.2, 30.1, 30.1, 30.0, 30.0, 29.8, 29.7, 29.7, 29.6, 28.1, 27.3, 27.2, 23.2, 18.3, 18.0, 14.3, 12.8, 12.5.

### Example 2: Peptide Synthesis in Solution

### Deprotection

The Fmoc protected tag (1 eq.) was dissolved in ethyl acetate (1%). Then diethylamine (4 eq.) was added and stirred overnight.

The TIPS protected tag (1 eq.) was dissolved in THF (1%). Then TBAF (2 eq.) was added and stirred for 2 h.

### Coupling

The deprotected tag was dissolved in ethyl acetate (1%). Then the Fmoc protected amino acid (Fmoc-AA-OH) (2 eq) and Oxyma (2 eq) was added and stirred for 1 min. Then N,N′-Diisopropylcarbodiimide (3 eq) was added and stirred overnight.

### Extraction

The reaction mixture was transferred into a separation funnel and washed with water (10 volume eq) and brine (10 volume eq). The tag stays in the organic phase and can be used for the next coupling step in solution.

1. A compound according to formula (I) 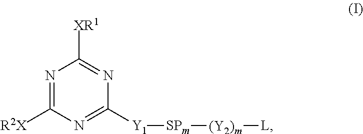
wherein,
X is selected from the group consisting of —O—(CH2)n-, wherein n is 0 to 4,
R1 and R2 are independently selected from the group consisting of C11-C25 alkyl,
SP is a spacer, particularly selected from the group consisting of saturated C4-C8 alkyl, unsaturated C4-C8 alkyl and substituted or unsubstituted C6-aromatic compound, in particular the substituted C6-aromatic compound is an alkyl substituted C6-aryl,
Y is a heteroatom, particularly selected from the group consisting of —O— and —NH—,
L is a linker for peptide synthesis, and
m is 0 or 1, particularly m is 1.

2. The compounds according to claim 1, wherein n is 0.

3. The compound according to claim 1, wherein R1 and R2 are independently from each other a branched or substituted C11-C25 alkyl.

4. The compound according to claim 1, wherein R1 and R2 are independently selected from the group consisting of C13-C25 alkyl.

5. The compound according to claim 1, wherein R1 is selected from the group consisting of C17-C25 alkyl.

6. The compound according to claim 1, wherein R2 is selected from the group consisting of C17-C25 alkyl.

7. The compound according to claim 1, wherein Y1 is —NH— and Y2 is —NH— or —O.

8. The compound according to claim 1, wherein Y1 is —NH— and Y2 is —NH—.

9. The compound according to claim 1, wherein SP is selected from the group consisting of saturated C4-C8 alkyl, unsaturated C4-C8 alkyl and substituted C6-aromatic compound.

10. The compound according to claim 1, wherein SP is selected from the group consisting of saturated C4-C8 alkyl, and substituted C6-aromatic compound.

11. The compound according to claim 1, wherein SP is selected from the group consisting of C4-C8 alkyl.

12. The compound according to claim 1, wherein the linker L selected from the group consisting of Rink, Sieber, Trityl, Ramage, Wang, Sasrin, HMPB, 2-chlorotrityl, MBHA, PAL, HMBA and HMPA.

13. The compound according to claim 1, wherein the linker L is selected from the group consisting of Ramage, Rink, HMPA, PAL and HMBA.

14. The compound according to claim 1, wherein the linker L selected from the group consisting of Rink, Sieber, Trityl, Ramage, Wang, Sasrin, HMPB, 2-chlorotrityl and MBHA.

15. The compound according to claim 14, wherein the linker L is selected from the group consisting of Rink, Sieber and Trityl.
